(19) United States
(12) Patent Application Publication
(10) Pub. No.: US 2007/0225791 A1
(43) Pub. Date: Sep. 27, 2007

(54) SURGICAL DEVICE FOR THE FORMATION BY A PRACTITIONER OF AT LEAST ONE IMPLANT

(76) Inventors: Audrey Molitor, Lille (FR); Mathieu Charret, Frelinghien (FR)

Correspondence Address: LADAS & PARRY LLP, 224 SOUTH MICHIGAN AVENUE, SUITE 1600, CHICAGO, IL, 60604, US

(21) Appl. No.: 11/688,357

(22) Filed: Mar. 20, 2007

(30) Foreign Application Priority Data
Mar. 21, 2006 (FR) 0602460

(51) Int. Cl. A61F 2/06

(52) U.S. Cl. 623/1.11

(57) ABSTRACT

A surgical device that allows the formation by the practitioner of at least one implant with a given configuration and made from a given bio-compatible material. The surgical device includes a panel and a first sheet. The The surgical device includes a panel and a first sheet. The panel is made from the bio-compatible material, and at least one dimension of the panel is greater than the corresponding dimension of the implant
The first sheet (2) that includes the trace of at least the contour of the implant, and the first sheet is placed on one face of the the panel and attached to the latter so that the practitioner is able to shape the implant by cutting the assembly composed of the combined first sheet and panel by following the trace. The trace can include several sets of cutting-out lines outlining several possible contours of the implant, and the first sheet can include several traces of implant contours corresponding to the treatments for different pathologies.

### BACKGROUND OF THE INVENTION

This present invention has as its subject a surgical device for the formation by the practitioner of at least one implant of a specified configuration.

During a surgical operation, in particular in visceral and uro-gynecological surgery, it is common for the practitioner, before the insertion of an implant, to first shape its contour to suit the morphology of the implant part of the patient and/or the state of the pathology to be treated.

The practitioner is usually in possession of an implant that is already formed according to a predetermined configuration designed in a general manner for the pathology. It is therefore firstly limited to the contour dictated by the implant and to the space available for it, and secondly is not helped to first shape the implant—frequently being of complex configuration—causing the practitioner to lose much valuable time and creating a risk of not achieving the final desired shape of the implant.

Furthermore, in order to meet the needs of the patients, the implants are sold separately in configurations that are predetermined for a given pathology, thus increasing the commercial range of implants to be supplied to, and to be stored by the distributors or the hospitals.

This present invention in particular has as its objective to overcome the aforementioned drawbacks by proposing a surgical device for the formation, by the practitioner, of at least one implant with a given configuration, and made from a given bio-compatible material, characterised in that it includes:
a panel made from the said material, and of which at least one dimension is greater than the corresponding dimension of the implant,
a first sheet with the trace of at least the contour of the implant,
and in that the said first sheet is placed on one face of the said panel and attached to the latter so that the practitioner is able to shape the implant by cutting the assembly composed of the first sheet and the associated panel by following the said trace.

Since the dimensions of the panel are equal to or greater than those of the implant, the practitioner possesses enough of the said material to configure the implant as he wishes. Furthermore, the practitioner is guided in cutting out the said assembly by following the trace of the contour of the implant, thus saving valuable time and preventing irremediable cutting errors.

The panel from which the implant is cut out is generally flat. It can be three-dimensional however, such as a panel that is pre-shaped and crimped for example. The resulting implant therefore has a flat or three-dimensional configuration.

The panel is made from a bio-compatible material, either a textile material of the knitted woven or non-woven type, such as a knitted polypropylene or polyester or polyamide or polylactic acid fabric for example, of L or D shape, or a polymer sheet, such as a calendered silicone sheet, for example.

The first sheet, with the trace, is placed on one face of the panel. Preferably, it is in a material that allows the trace to be seen clearly, and that also allows the panel to be seen, in particular where it concerns an opaque or transparent or translucent sheet. The trace of at least the contour of the implant is preferably created with a biocompatible ink.

In an implementation variant, the first sheet is one of the two surfaces of an envelope containing the said panel. The other face of the envelope, opposite to the first sheet with the trace of at least the contour of the implant, is called a second sheet, and is made from a material which can be the same as that from which the first sheet is made, or different from the latter. It is the assembly composed of the first and second sheet and the panel that is cut out by the practitioner. The panel is associated with the first sheet because it is contained in the said envelope even though it is placed loosely inside the latter.

In an implementation variant, the first and the second sheet, forming both surfaces of the envelope, are joined over all or part of their periphery around the panel by high-frequency welding, ultrasound welding, heat welding, glueing or any other equivalent means.

Preferably, the first and second sheets, forming the two surfaces of the envelope, are rectangular and joined on two sides and possibly a third side in order firstly, to provide lateral support during the cutting out, and thus facilitating the shaping of the implant, and secondly to leave at least one opening, formed in this case on the non-welded side, through which the panel can pass or be accessible so that one or more ties can be attached to the material of the panel, and left protruding via the said opening, outside of the envelope, thus preventing them from being cut accidentally by the practitioner when using the cutting tool. These ties are threaded by the practitioner onto instruments that he uses for the insertion and the positioning of the implant in the zone of the surgical operation.

The said first and second rectangular sheets can also be welded over the whole of their periphery and freely enclose the said panel. Retention is thus improved because all the sides are welded.

In an implementation variant, the first sheet is welded on all or part of its periphery around the said panel by high-frequency welding, ultrasound welding, heat welding or glueing. Preferably, the said first sheet is welded on three sides of the said panel. This has the same advantages as in the previous variant, when the envelope contains the panel, namely lateral retention and the ability to attach ties.

In this variant, the panel is inseparable from the said first sheet, in contrast to the previous variant where the panel was placed freely inside the said envelope, and it can be replaced by another panel, in a different material for example.

In an implementation variant, the said first sheet is placed on one face of the said panel and attached to the latter by means of a bio-compatible, repositionable adhesive. The adhesive generally covers all or part of the surface of one of the faces of the said first sheet. The adhesive is said to be repositionable when it can be removed from the support to which it is applied, without damaging the said support, and can be repositioned if necessary, on the same or another support, with its adhesion properties remaining sufficient to attach it firmly to the said support.

Advantageously, during the cutting out of the assembly composed of the first sheet and the panel, the first sheet remains attached to the panel. The practitioner can unstick the first sheet and then stick it down again in a different position on a face of the said panel as many times as necessary. After cutting out the said assembly by following the trace, the practitioner finishes the implant by removing the first sheet from the panel.

Naturally, the assembly formed by the first sheet attached directly to the said panel or to a second sheet with which it forms an envelope freely containing the said panel is not limited to a rectangular shape, and can take any shape to suit the application concerned.

In an implementation variant, the trace includes several sets of cutting-out lines outlining several possible contours of the implant. The practitioner is thus guided to configuration variants for a given implant, designed for a given type of pathology, thus enabling him to save time, and affording a certain convenience by enabling him to avoid cutting-out errors and avoiding the need to supply each implant variant separately.

In an implementation variant, the said first sheet includes several traces of implant contours corresponding to the treatments for different pathologies. For example, the said first sheet can include two traces with several sets of cutting-out lines for each of these, with each trace being intended to form an implant for the treatment of a given pathology. Advantageously, this arrangement avoids the need to supply each implant separately according to the different pathologies, whether at production level (cutting out by the operators, packaging, sterilisation, etc.) or during commercial distribution.

Once fashioned, the surgical device of this present invention is then packed into two sachets, also called a double-sachet, and then sterilised in ethylene oxide or by gamma radiation for example.

In an implementation variant, the surgical device includes at least one tie attached to the panel, intended to act as an attachment device for the implant.

For the practitioner, the said attachment device facilitates the insertion and the positioning of the implant in the part of the patient to be treated, by being attached easily to an insertion-type instrument. The said tie can take the form of a loop for example, or can be a staple.

The said tie is preferably attached to the part of the panel protruding from or accessible from the opening formed by a unwelded or unstuck part of the envelope or of the first sheet, so that the practitioner is not impeded or in danger of cutting the said tie when cutting out the implant. For the case where one of the faces of the first sheet is coated on all or part of its surface with a repositionable adhesive, preferably the said face is attached to a face of the said panel so as not to cover the said tie and to leave it free.

In an implementation variant, the said trace corresponds to the contour of an implant intended for the treatment of a rectocele, a cystocele, a hernia or an incisional hernia.

In an implementation variant, and in a characteristic manner, the surgical device includes a panel protruding from the first sheet, in particular by extensions that are intended to form attachment tabs for the implant.

The said attachment tabs can thus protrude from the first sheet in particular to form a length that is greater than the length of the side of the first sheet through which they protrude.

### BRIEF DESCRIPTION OF THE DRAWING FIGURES

This present invention will be understood more clearly on now reading about types of surgical device, given by way of non-limiting examples, and illustrated by the following figures:

FIG. 1 is a schematic plan view, from above, of a first example of a surgical device according to this present invention;

FIG. 2 is a schematic plan view, from above, of a second example of surgical device according to this present invention;

### DETAILED DESCRIPTION OF THE PREFERRED EMBODIMENTS

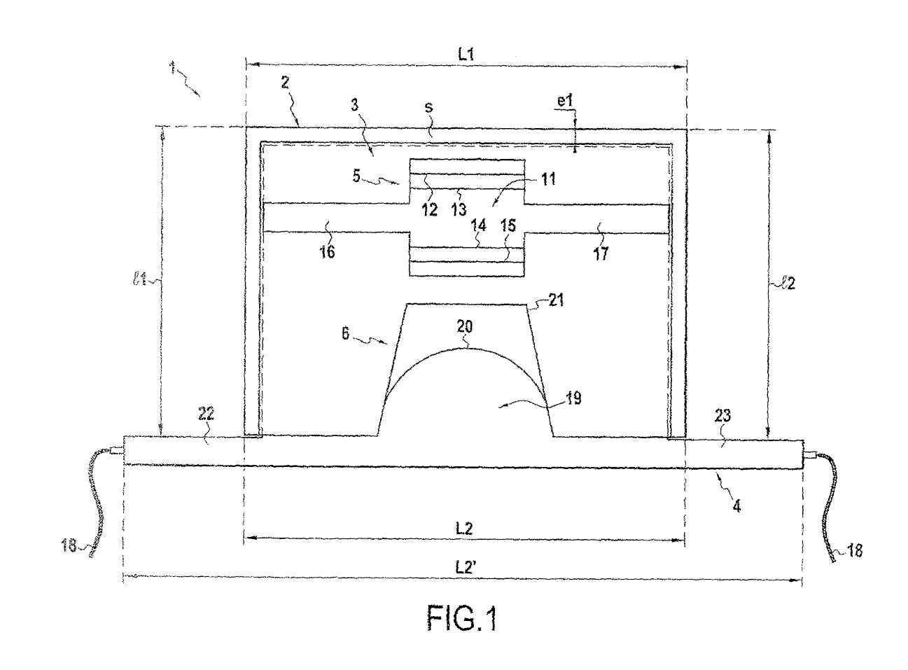

In FIG. 1, the surgical device 1 according to this present invention takes the form of a rectangular envelope 2, which includes a flat panel 4 placed loosely inside the latter. The envelope 2 has two faces, in the form of a first sheet 3 and a second sheet not visible in FIG. 1, since it has the same dimensions as the first sheet 3 and is located at the back of the envelope 2. The first sheet 3 and the second sheet forming the envelope, are heat-welded on two small sides l1, l2 and their large side L1, along weld lines, and over the width e1. Preferably, e1 measures a few millimetres, and more particularly e1 measures 5 millimetres. The panel 4 overhangs the large unwelded side L2 in the form of two extensions 22, 23 with a total width L2′ that is greater than length L2. These extensions 22, 23 act as attachment tabs for the implant on the tissue and/or the organs of the patient.

The first sheet 3 has printed on one of its faces, but visible from the outside, two traces of implant contours corresponding to the treatment of two different pathologies. The first pathology is the rectocele, and corresponds to the first trace 5. The second pathology is the cystocele, and corresponds to the second trace 6. Each trace 5,6 includes several sets of cutting-out lines, in order to offer the practitioner different flat configuration variants for shaping the implant.

During the use of the surgical device 1, the practitioner, according to the pathology to be treated, namely a rectocele or a cystocele, and the configuration that he wishes to give to the implant, cuts out the assembly formed of the first sheet 3 and the second sheet together with the panel 4 by following the contour of the chosen cutting-out lines, and thus forms a configuration implant that is designed for the pathology and for the patient, and that is ready for use.

Preferably, the practitioner begins cutting out the said assembly starting from a small side l1 and/or l2, with the large side L1 then providing lateral retention to preserve the attachment of the panel 4 to the envelope 2 at least at the beginning of the cutting-out process. The practitioner performs the cutting out of the said assembly by means of conventional or electric scissors or shears, or any other equivalent means.

When the cutting out of the said assembly has been completed over all the contour of the chosen configuration, then since panel 4 is placed freely in the envelope 2, the implant of the configuration chosen by the practitioner is removed from the said assembly and is ready to be inserted and placed in the part of the patient to be treated.

The two traces 5, 6 shown in FIG. 1 correspond to implants that have a central portion, respectively 11 for the rectocele 5 and 19 for the cystocele 6, whose dimensions can be adjusted by means of cutting-out lines. In the case of the first trace 5 for the rectocele, the surface area of portion 11 can be adjusted by means of cutting-out lines 12, 13, 14 and 15. In addition, the first trace 5 outlines two strips 16, 17 extending away from each other and from the central portion 11. In the case of the second trace 6 for the cystocele, the practitioner can choose a rounded shape for the central portion 19 by following cutting-out line 20, or a trapezium shape by following cutting-out line 21, according to the dimensions of the surgical operation zone.

In the case of trace 6 for the cystocele, the panel 4 overhangs the envelope 2 on the second large side L2, and includes two lateral extensions 22, 23 which extend outwards from the central portion 19, beyond the said second large side L2. Two ties 18 are crimped to the two ends of the said extensions and form attachment devices in the form of loops that can be attached by the practitioner to instruments of the insertion type to assist with the insertion and positioning of the implant in the patient. In this implementation example, the ties 18 are braids.

Preferably, the panel 4 is made from a bio-compatible textile material, and more particularly in a knitted polypropylene fabric. Since the panel 4 is placed freely in the envelope, it is possible to replace it with a panel that has the same dimensions and made from a different bio-compatible material such as an implantable silicone-impregnated polyester knitted fabric for example, which not only provides the practitioner with different configurations for the shaping of an implant by means of traces, possibly consisting of cutting-out lines borne on one of the faces of the envelope, but also with the choice of the material of which they are made, through the ability to substitute the panel in the said envelope.

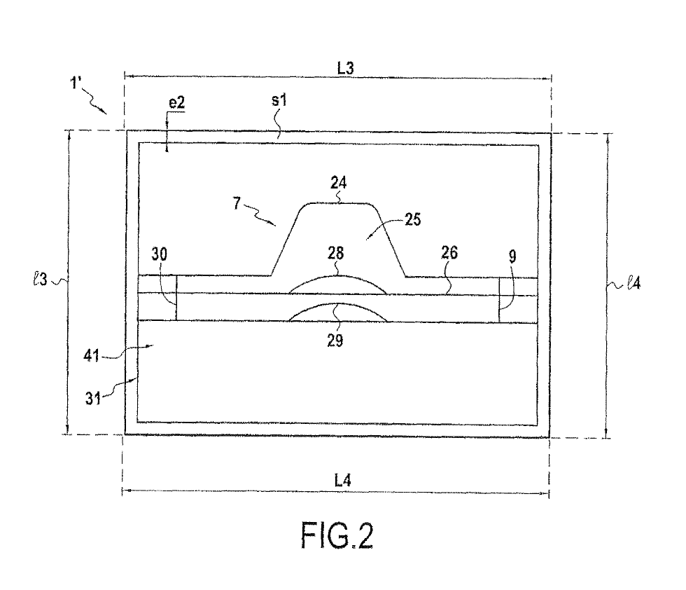

The second example of a surgical device 1′ according to this present invention, shown in FIG. 2, is of simpler design than the device 1. It consists only of a first sheet 31 and rectangular panel 41. The first sheet 31 includes a single trace 7 for an implant contour corresponding to the treatment of a pathology. In this particular case, it concerns the cystocele. The trace 7 borne on the first sheet 31 includes numerous cutting-out lines, namely 9, 24, 26, 28, 29, 30. The practitioner can adjust the treatment of the cystocele by cutting along cutting-out line 28 or 29, which creates a notch in portion 25, which enables him, in the case of this pathology, to leave free the neck of the bladder and thus to treat only the prolapse. The first sheet 31 is affixed over the whole of its periphery, composed of two small sides l3, l4 and the two large sides L3, L4, directly onto the panel 41 over width e2, along weld line s1. There is no fixed tie on the panel 41. In this case, in contrast to the first implementation example, it is not possible to change the panel 41 once the latter has been attached to the first sheet. The practitioner forms the implant by cutting the panel 41 and the first sheet 31 along trace 7 by means of the cutting-out lines that he has chosen. The implant can be removed easily from the panel 41 and from the first sheet 31 because the welding exists only over width e2. This too has some lateral retention at least at the beginning of the cutting-out process. In fact, when the practitioner begins the cut out on one of the small sides, the large sides L3, L4 hold the panel 41 and the first sheet 31 laterally, and vice versa when the cut out starts from one of the lengths L3 or L4.

1. A surgical device for the formation by the practitioner of at least one implant, of a given configuration and made from a given bio-compatible material, the surgical device comprising:
a) a panel in the said material, and of which at least one dimension is greater than the corresponding dimension of the implant,
b) a first sheet, which is not intended to be implanted, and which includes the trace of at least the contour of the implant, and
wherein said first sheet is placed on one face of the said panel and attached to the latter so that the practitioner forms the implant by cutting the assembly composed of the first sheet and the associated panel by following said trace, and then withdrawing the cut portion of said first sheet.

2. A device according to claim 1, wherein said first sheet is one of the two surfaces of an envelope containing said panel.

3. A surgical device according to claim 2, wherein both surfaces of the envelope are attached over all or part of their periphery around the panel by high-frequency welding, ultrasound welding, heat welding, or gluing.

4. A surgical device according to claim 1, wherein said first sheet is welded over all or part of its periphery around said panel by high-frequency welding, ultrasound welding, ultrasound welding, heat welding, or gluing.

5. A surgical device according to claim 1, wherein said first sheet is placed on one face of said panel and attached to the latter by means of a bio-compatible, repositionable adhesive.

6. A surgical device according to claim 1, wherein said trace includes several sets of cutting-out lines, outlining several possible contours of the implant.

7. A surgical device according to claim 1, wherein said first sheet includes several traces of implant contours corresponding to the treatments for different pathologies.

8. A surgical device according to any of the previous according to claim 1, including at least one tie attached to the panel, intended to act as an attachment device for the implant.

9. A surgical device according to claim 1, wherein said trace corresponds to the contour of an implant intended for the treatment of a rectocele, a cystocele, a hernia or an incisional hernia.

10. A surgical device according to claim 1, wherein said panel overhangs the first sheet, in particular in the form of extensions intended to form attachment tabs for the implant.
